(19) United States
(12) Patent Application Publication
(10) Pub. No.: US 2026/0256588 A1
(43) Pub. Date: Sep. 3, 2026

(54) MEDICAL DEVICES, SYSTEMS, AND RELATED METHODS

(71) Applicant: Xenter, Inc., Draper, UT (US)

(72) Inventor: Richard J. Linder, Sandy, UT (US)

(73) Assignee: Xenter, Inc., Draper, UT (US)

(21) Appl. No.: 19/656,357

(22) Filed: Apr. 23, 2026

Related U.S. Application Data
(63) Continuation of application No. PCT/US24/52633, filed on Oct. 23, 2024.
(60) Provisional application No. 63/592,398, filed on Oct. 23, 2023.

(51) Int. Cl. A61F 2/24; A61B 8/00; A61B 8/04; A61B 8/12; A61M 25/00; A61M 25/09

(52) U.S. Cl. CPC A61F 2/2472; A61B 8/04; A61B 8/12; A61B 8/565; A61F 2/2427; A61M 2025/0002; A61M 2025/09175

(57) ABSTRACT

Various embodiments of a medical device, a medical device system, and associated methods are set forth herein. In one embodiment, a system includes a guidewire having a pair of pressure sensors in a distal section of the guidewire. A control unit may be selectively coupled with a proximal end of the guidewire and in communication with the sensors. An external computing device may be placed in wireless communication with the control unit. A monitor may be placed in wireless communication with the external computing unit and configured to display pressure data obtained by the pair of pressure sensors. In one embodiment, an imaging device may be placed between the two sensors and be used to determine a size of a physiological structure (e.g., the size of a replacement heart valve).

### CROSS-REFERENCE TO RELATED APPLICATIONS

The present application is a continuation of Patent Cooperation Treaty Application PCT/US24/52633, filed on Oct. 23, 2024, entitled MEDICAL DEVICES, SYSTEMS, AND RELATED METHODS, which claims the benefit of U.S. Provisional Application No. 63/592,398, entitled MEDICAL DEVICES, SYSTEMS, AND RELATED METHODS, filed on Oct. 23, 2023, the disclosures of each of which are incorporated by reference herein in their entireties.

### BACKGROUND

The present disclosure relates generally to medical devices incorporating sensors, as well as systems and methods which may incorporate such devices. Additionally, the present disclosure relates to devices and systems for providing information to a healthcare practitioner who may be utilizing such devices, systems or practicing associated methods.

In one, non-limiting example, such medical devices may include intraluminal devices, such as guidewires, having one or more sensors for measuring of one or more physiological parameters and/or for imaging.

Guidewire devices are often used to lead or to guide catheters or other interventional devices to a targeted anatomical location within a patient's body. For example, guidewires may be passed into and through a patient's vasculature in order to reach the target location, which may be, for example, at or near the patient's heart or brain. Radiographic imaging is conventionally utilized to assist in navigating a guidewire to the targeted location. Guidewires are available with various outer diameter sizes. Widely utilized sizes include 0.010, 0.014, 0.016, 0.018, 0.024, and 0.035 inches in diameter, for example, though they may also be smaller or larger in diameter.

In some instances, a guidewire may be used to gather physiological information from within a patient. For example, so-called “pressure wires” conventionally incorporate a single pressure sensor to detect the blood pressure within a blood vessel of a patient.

In many instances, a guidewire is placed within the body during the interventional procedure so that it can be used to guide one or more catheters or other interventional devices to the targeted anatomical location. For example, a catheter can be guided to a targeted location and, once in place, be used to image the targeting location, to aspirate clots or other occlusions, or to deliver drugs, stents, embolic devices, radiopaque dyes, replacement valves, or other devices or substances for treating the patient.

These types of interventional devices can include sensors located at the distal end in order to provide added functionality to the device. For example, intravascular ultrasound (IVUS) is an imaging technique that utilizes a catheter with an ultrasound imaging sensor attached to the distal end. Ultrasound may be utilized to image within targeted vasculature (typically the coronary arteries).

There are several challenges associated with using sensors with intraluminal devices. For example, such interventional devices have very limited space to work in, given the stringent dimensional constraints involved. Moreover, integrating the sensors with the interventional device in a way that maintains effective functionality can be challenging.

The use of such interventional devices can also be challenging due to the need to manage several long lengths of wires and other components, including guidewires, power cables, data wires, and the like. Care must be taken with respect to what is allowed in the sterile field and when certain devices or components can be removed. Additional staff is often required simply to manage such wires, cables, and components.

As such, there is an ongoing need for improved medical devices that effectively integrate sensors and can help provide data in a more efficient manner and/or provide data previously unobtainable in a practical manner.

### SUMMARY

Medical devices, medical systems, and various methods are set forth in accordance with various embodiments of the present disclosure. In accordance with one embodiment, a medical system comprises: a guidewire having a pair of pressure sensors in a distal section of the guidewire; a control unit selectively coupled with a proximal end of the guidewire; an external computing device in wireless communication with the control unit; and a monitor in wireless communication with the external computing unit configured to display pressure data obtained by the pair of pressure sensors.

In one embodiment, the external computing device is in communication with a healthcare facility network.

In one embodiment, the external computing device is in communication with an external database.

In one embodiment, the pair of pressure sensors are spaced between approximately 9 centimeters (cm) and approximately 10 cm from each other in a center-to-center fashion.

In one embodiment, the guidewire includes a coiled section at a distal portion of the guidewire that extends from a longitudinal portion of the guidewire.

In one embodiment, a distal-most sensor of the pair of sensors is positioned a distance of between approximately 2 cm and approximately 2.5 cm from a line that is tangent to a proximal-most portion of the coiled section and is perpendicular to the longitudinal portion to the center of the distal-most sensor.

In one embodiment, the guidewire further includes and imaging device positioned longitudinally between the pair of pressure sensors.

In one embodiment, the imaging device includes an ultrasound transducer.

In another particular embodiment, a medical device comprises a guidewire having a pair of pressure sensors in a distal section of the guidewire and an imaging device associated with the guidewire, the imaging device positioned longitudinally between the pair of pressure sensors.

In one embodiment, the imaging sensor includes at least one ultrasound transducer.

In one embodiment, wherein the pair of pressure sensors are spaced between approximately 9 centimeters (cm) and approximately 10 cm from each other in a center-to-center fashion.

In one embodiment, the guidewire includes a coiled section at a distal portion of the guidewire that extends from a longitudinal portion of the guidewire.

In one embodiment, a distal-most sensor of the pair of sensors is positioned a distance of between approximately 2 cm and approximately 2.5 cm from a line that is tangent to a proximal-most portion of the coiled section and is perpendicular to the longitudinal portion to the center of the distal-most sensor.

In another embodiment of the present disclosure, a method includes: positioning a guidewire in a patient such that a first sensor of the guidewire is positioned in a left ventricle and a second sensor is positioned in an aorta; measuring a first aortic pressure with a first sensor and a first ventricular pressure with the second sensor; positioning a replacement valve at a location between the first sensor and the second sensor; measuring a second aortic pressure with the first sensor and a second ventricular pressure with the second sensor; and determining whether to adjust the replacement valve based on a comparison of data obtained from the first aortic pressure, the second aortic pressure, the first ventricular pressure, and the second ventricular pressure.

In one embodiment, the method further includes adjusting the replacement valve.

In one embodiment, the method further includes measuring a third aortic pressure with the first sensor and a third ventricular pressure with the second sensor, and determining whether to further adjust the replacement valve based on a comparison of data obtained from the first aortic pressure, the second aortic pressure, the third aortic pressure, the first ventricular pressure, the second ventricular pressure, and the third aortic pressure.

In one embodiment, measuring the second aortic pressure and the second ventricular pressure is performed without removing the second sensor from the left ventricle subsequent the measuring of the first aortic pressure and the second aortic pressure.

In one embodiment, determining whether to adjust the replacement valve based on a comparison of pressure data obtained from the first aortic pressure, the second aortic pressure, the first ventricular pressure, and the second ventricular pressure includes comparing the pressure data to information in a database of previously-obtained data.

In one embodiment, the method further includes determining a size of the replacement valve using an imaging device of the guidewire located between the first sensor and the second sensor.

Elements, components, features, and acts of one embodiment may be combined or included with the elements, components, features, or acts of another embodiment without limitation.

### BRIEF DESCRIPTION OF THE DRAWINGS

The foregoing and other advantages of the invention will become apparent upon reading the following detailed description and upon reference to the drawings in which:

FIG. 1 illustrates a guidewire system according to an embodiment of the present disclosure;

FIG. 2 is an enlarged, detail view of a distal end of a guidewire in accordance with an embodiment of the present disclosure;

FIG. 3 is an enlarged, detail view of a proximal end of a guidewire in accordance with an embodiment of the present disclosure;

FIG. 4 is a partial cross-sectional view of a sensor mounting in a portion of a guidewire in accordance with an embodiment of the present disclosure;

FIG. 5 depicts a distal portion of a guidewire disposed within a patient's heart in accordance with an embodiment of the present disclosure;

FIG. 6 is a plan view of a controller removably coupled with a proximal portion of the guidewire in accordance with an embodiment of the present disclosure;

FIG. 7 is an enlarged, detail view of a distal end of a guidewire in accordance with another embodiment of the present disclosure

FIG. 8 is a flow diagram illustrating a method in accordance with an embodiment of the present disclosure.

### DETAILED DESCRIPTION

Various embodiments described herein are directed toward the incorporation of electronic devices (e.g., sensors and transducers) into medical devices, systems incorporating such medical devices, and related methods.

In some embodiments, devices associated with cardiovascular, neurovascular, and endovascular procedures are provided having sensors integrated therewith. For example, guidewires or catheters may include sensors, transducers or other electronic or optical components integrated into the structure for detecting, imaging or measuring physiological data (e.g., pressure, flow rate, etc.), providing imaging data (e.g., ultrasound images), and providing that data to a healthcare provider in real time during an associated procedure.

In some embodiments, other sensors or electronic elements are associated with the device. For example, sensors configured to detect the presence of biological components may be incorporated into or otherwise associated with the device. In some embodiments, a transceiver unit having an antenna structure may be associated with the device for providing wireless transmission of data.

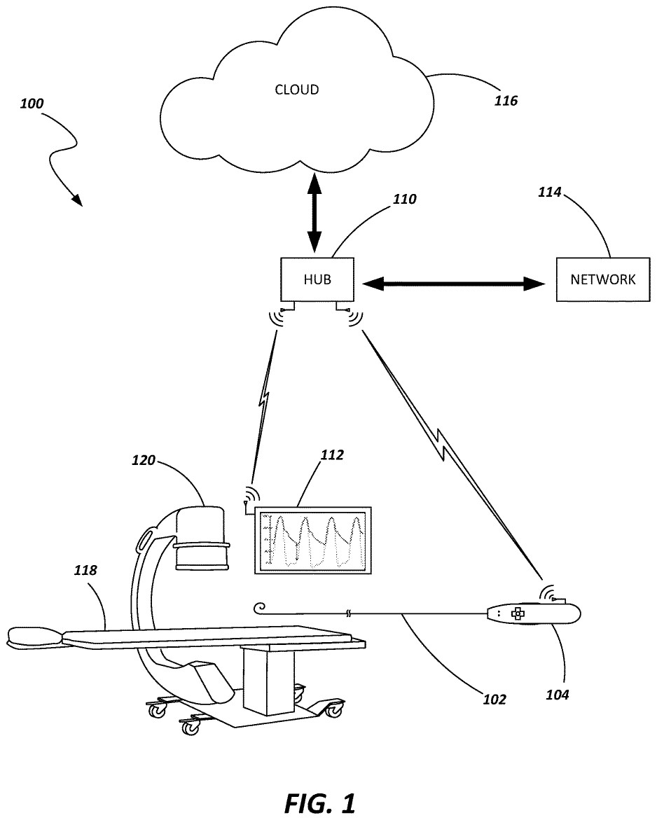

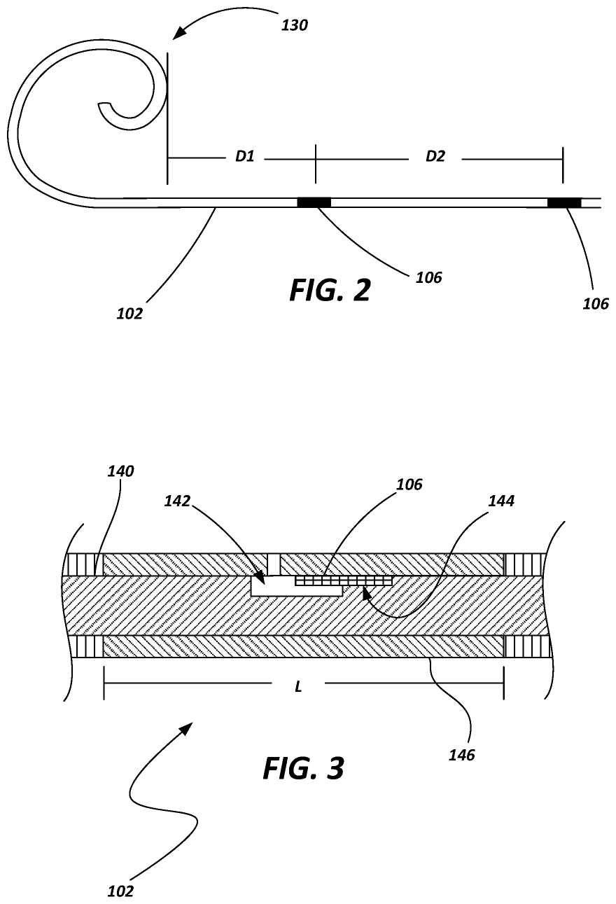

Referring to FIG. 1, a guidewire system 100 is illustrated according to an embodiment of the present disclosure. As shown, the guidewire system 100 includes a guidewire 102, a proximal device which, in some embodiments, may include a control unit 104 for providing power to, and communication with, sensors or other electronic or optical components associated with the guidewire 102. The system 100 further includes a plurality of sensors 106 (see, e.g., FIGS. 2 and 3) associated with a distal end of the guidewire 102. The control unit 104 may include, for example, a power source (e.g., a battery), a data signal processor, a memory device, and a transmitter/receiver (referred to herein as a transceiver). In some embodiments, such components may be disposed, entirely or partially, within a body or housing of the control unit 104.

The system 100 may further include an external computing device 110 (also referred to as a “hub”). The external computing device 110 may include, e.g., a stationary or handheld computer, a stationary or handheld display, a tablet computer, a smart phone, or other input and/or output device. In one embodiment, as depicted in FIG. 1, the external computing device 110 may be in wireless communication with the control unit 104. Any of a variety of wireless protocols may be utilized (e.g., Bluetooth, Zigbee, Wi-Fi, etc.).

The system may further include a monitor 112, or a “boom” used by medical personnel during a procedure to review data and information relating to the procedure and the status of the patient. The monitor 112 may be in wired or wireless communication with the external computing device 110 so as to display information obtained by the sensors 106 or other electronic or optical components associated with the guidewire 102. For example, if the guidewire were being used in association with a transcatheter aortic valve implantation (or replacement)—TAVI or TAVR—procedure, the sensors 106 may detect or determine a first pressure in the left ventricle and a second pressure in the aorta. Each of the sensors 106 each provides a signal representative of the obtained pressures to the control unit 108, which in turn relays them (either as they are or as a modified signal) to the external computing device 110. The external computing device 110 then relays the pressure data to the monitor 112 and presents in a recognizable form (e.g., as number and/or in a wave form) so that an interventional cardiologist may review the sensed pressures and determine if a valve replacement is necessary—or when measuring after the initial placement of a new valve, determine if subsequent actions need to be taken (such as reseating the valve to eliminate or reduce regurgitation).

The external computing device 110 is also in communication with the hospital's (or other healthcare facility's) computing system or network 114 for access to, for example, electronic healthcare records (EHRs) which may be relevant to the current procedure. Access to such information may be beneficial, for example, to consider a specific patient's health history as it pertains to the instant procedure. Further, the external computing device 110 may be in communication with a global cloud 116 or database having information relating to the instant procedure. The hospital computing system 114 and the cloud 116 may each be coupled with the external computing device 110 through wireless or through wired connections.

In one example, the cloud 116 may contain information relating to similar procedures including information relating to individuals in a similar demographic as the patient undergoing an instant procedure, their response to different interventions, their pressure or flow rates during a similar procedure, and other relevant data. The global cloud 116 may include computing ability to implement machine learning (or artificial intelligence) to apply the information within the global cloud to a specific procedure in light of the data being collected during the procedure. For example, a comparison of a pressure curve associated with the aortic pressure with the pressure curve of the left ventricular pressure may yield an index that is useful in determining whether an interventional act is required. In some embodiments, the index may be based strictly on a direct comparison of such pressures. However, in some embodiments, the index may be based on a dynamic analysis of the pressure curves, the past health history of the patient (e.g., as obtained from the EHR), data associated with the diagnosis and procedure outcomes of other individuals that may satisfy certain health and/or demographic criteria (e.g., age, race, weight, other diagnosed conditions, etc.). Thus, the index can be a dynamic tool to more accurately determine actions to be taken (or not taken) during a specific procedure as the procedure is being conducted.

It is noted that the system 100 may be defined to include certain basic elements (e.g., the guidewire 102 including its sensors 106, the control unit 104, and the external computing device 110), or it may be defined to include additional elements including the hospital network 114 and/or the global cloud 116). The system 100 may additionally include other components including those conventionally found in a catheterization lab, such as the monitor 112, a patient bed 118, an imaging device 120 for providing CT, X-ray, fluoroscopy, or other imaging information during the procedure. It is noted that the monitor 112 may be coupled with the imaging device 120 and may be configured to show imaging information as well as physiological information and that such information may be displayed simultaneously or individually as selectively determined by a practitioner.

Referring briefly to FIG. 2, a distal section of the guidewire 102 is depicted. The distal section includes curved (e.g., spiraled) or coiled section 130, sometimes referred to as a “pigtail”, that is configured to engage with a portion of the patient's anatomy, effectively anchoring the guidewire in a desired position in an atraumatic fashion. The portion proximal of the coiled section 130 further includes two or more sensors 106 that are longitudinal spaced along a length of the guidewire 102. In one embodiment, the sensors 106 may be configured as pressure sensors (e.g., piezoelectric or capacitive-type pressure sensors). In one embodiment, the distal most sensor 106A may be positioned at a location a distance D₁ taken from a tangent line of the proximal most portion of the coiled section 130 and which extends perpendicular to the length of the guidewire 102 as indicated in FIG. 2. In one embodiment, the distance D₁ may be between approximately 1 cm and approximately 3.5 cm. In one particular embodiment, the distance D₁ may be between approximately 2 cm and approximately 2.5 cm. Such measurements referred to above being measured from the tangent line to the center of the distalmost sensor 106A as depicted in FIG. 2.

The sensors are spaced apart a distance “D₂” so that one may be positioned in a patient's left ventricle while the other is positioned within the patient's aorta. In one embodiment, distance D₂ may be approximately 9 centimeters (cm) apart. In one embodiment, distance D₂ may be approximately 10 cm apart. In one embodiment, distance D₂ may be approximately 11 cm apart In another embodiment, distance D₂ may be between approximately 8 cm and approximately 11 cm apart. In another embodiment, distance D₂ may be between approximately 7 cm and approximately 12 cm apart. Such measurements referred to above being measured from the center of one sensor to the center of the adjacent sensor as depicted in FIG. 2.

In one embodiment, the distance D₁ may be approximately 2.5 cm while the distance D₂ may be approximately 10 cm. In another embodiment, the distance D₁ may be approximately 2 cm while the distance D₂ may be approximately 10 cm.

Referring now to FIG. 3, a cross-sectional view of a sensor 106 mounted in the guidewire 102 is provided. The guidewire 102 includes a core wire 140 which may be formed of a metallic material such as, for example, stainless steel or titanium. The core wire 104 has a portion removed to form a pocket 142 or a void. The pocket 142 may be formed by machining, laser ablation, or other appropriate manufacturing techniques. Next to the pocket 142, a shelf or a stepped region 144 is formed within the core wire 140. A first portion of the sensor 106 is attached to the stepped region 144 (e.g., such as by adhesive) such that another portion of the sensor 106 is cantilevered into the pocket 142, leaving a gap or a space between the underside of the cantilevered sensor portion and the bottom of the pocket 142 formed in the core wire 140.

A housing 146 is positioned over the core wire 104, the sensor 106 and the pocket 142. The housing 146 may be formed of a metallic material such as, for example, stainless steel or titanium. In one embodiment, the housing exhibits a longitudinal length “L” of approximately 0.5 cm. In other embodiments, the length L may be between approximately 1.0 cm and 0.25 cm. The housing 146 may help maintain the position of the sensor 106 or otherwise secure the sensor 106 to the core wire 104. The housing 146 additionally provides support to the core wire 104 in the region where the pocket 142 and stepped region have been formed such that the core wire 140 may withstand bending forces applied in that region of the core wire 140 when the guidewire 102 is being navigated through a tortuous path of a patient's anatomy. An opening 147 is formed in the housing 146 to provide fluid communication between the pocket 142 and the external environment in which the guidewire 102 is placed. Thus, for example, if the guidewire is positioned such that the sensor 106 is located within a patient's aorta, the pocket is in fluid communication with the blood that is flowing within the aorta, enabling the sensor 106 to determine the blood pressure at that location.

Another material layer 148 may be positioned about the core wire 140 in locations adjacent to the housings 146 to provide a common diameter and provide a smooth outer surface for the guidewire 102 and eliminate any abrupt transitions that might otherwise occur (e.g., a stepped transition that might occur along the length of the core wire 140 and the housings 146). The material layer 148 may include, for example, a polymer material such as polyimide. In some embodiments, the material layer 148 may extend over the housings 146, while still leaving an opening (e.g., associated with opening 147) for fluid communication into the pocket 142.

The configuration of the cantilevered sensor 106 within the pocket enables the sensor 106 to avoid or minimize inaccurate pressure readings that might otherwise be induced by the bending of the core wire 140. In other words, the bending of the core wire 140 at the location of the sensor 106 does not subject the sensor to a false reading because the cantilevered portion is “free” from the bending and does not register such bending forces as it would if the entire sensor were adhered or otherwise attached to the core wire 140.

The resulting guidewire 102 may have a size such that the outer diameter (e.g., after application of other outer members and/or coatings) is about 0.008 inches to about 0.040 inches, though larger or smaller sizes may also be utilized depending on particular application needs. For example, particular embodiments may have outer diameter sizes corresponding to standard guidewire sizes such as approximately 0.010 inches, 0.014 inches, 0.016 inches, 0.018 inches, 0.024 inches, 0.035 inches, 0.038 inches, or other such sizes common to guidewire devices. The wire 102 may be formed from materials comprising stainless steel or other metal or alloy having similar appropriate properties.

While the guidewire 102 has been primarily described as including pressure sensors, it is noted that other sensors may be used in addition to such pressure sensors or in the alternative of such pressure sensors. For example, the sensors may additionally, or alternatively, be configured to determine flow rate or to sense the presence of biological components or measure physiological parameters in the targeted anatomical location (e.g., in the blood). Example biological components that may be detected/measured include sugar levels, pH levels, CO2 levels (CO2 partial pressure, bicarbonate levels), oxygen levels (oxygen partial pressure, oxygen saturation), temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), and/or toxins (e.g., lead, ethanol).

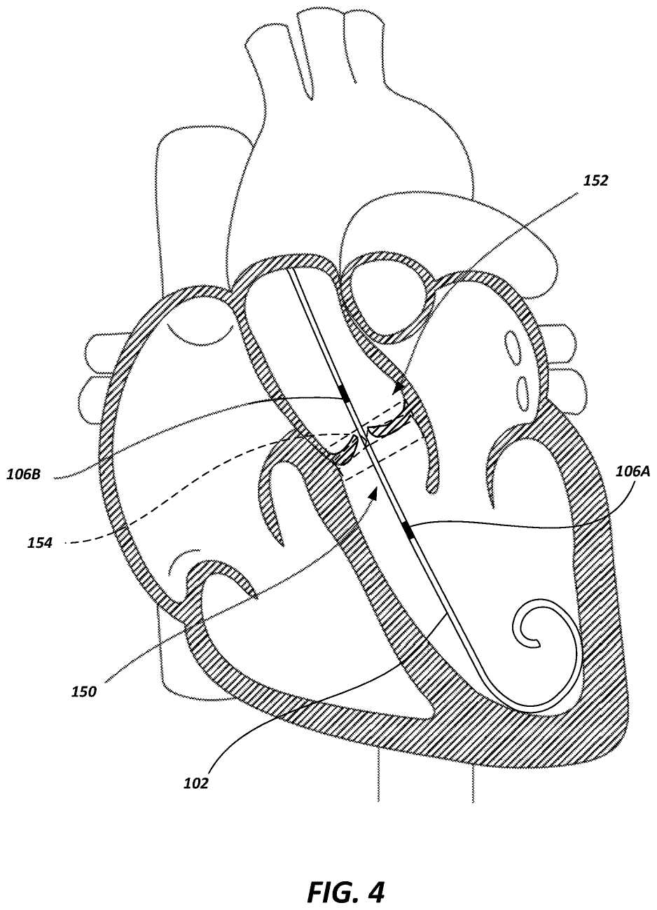

Referring briefly to FIG. 4, the distal section of the guidewire 102 is shown to be positioned within a patient's anatomy such that a first sensor 106A is located within the patient's left ventricle 150 and a second sensor is positioned within the patient's aorta 152. As noted above, this enables pressure readings to be taken simultaneously at both locations to determine, for example, the level of regurgitation that a patient is experiencing across the aortic valve. The pressure readings may be displayed on a monitor 112 (FIG. 1) for a practitioner to review and determine what, if any, action should be taken. The guidewire 102, thus, may be used in diagnosis of a patient's condition, but also used after the diagnosis to guide a catheter delivering a replacement valve to the target location if needed or desired. Subsequent the delivery and placement of a new valve 154 (shown in dashed lines), the guidewire 102 may be again used to measure pressures in the left ventricle and the aorta to determine if the valve implantation/replacement was successful, or if repositioning of the valve may be desirable. Thus, the guidewire 102 may be used throughout the valve replacement procedure without removal of the guidewire 102 until the practitioner is satisfied with the placement and securement of the new valve.

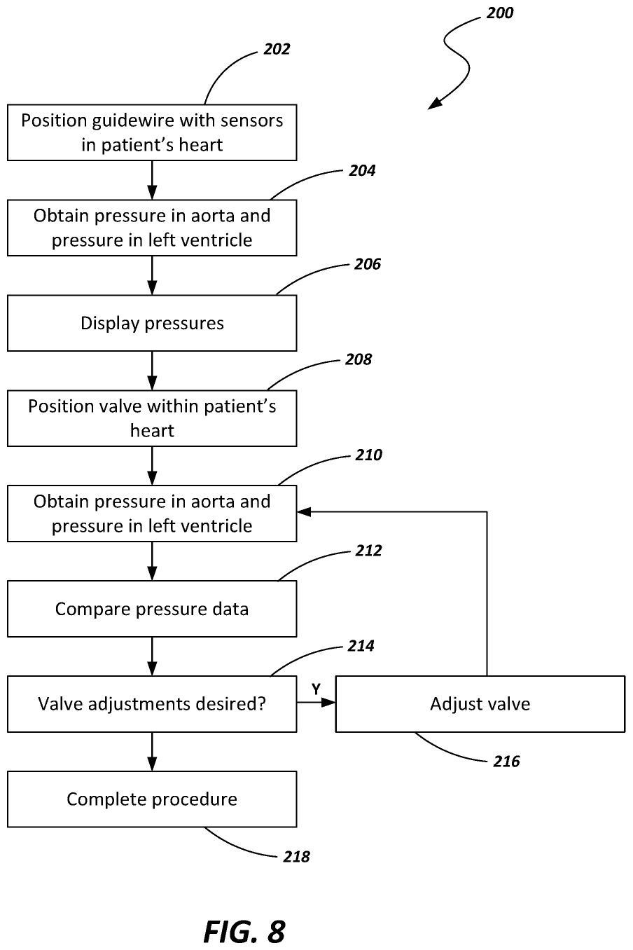

Referring briefly to FIG. 8 in association with FIGS. 1, 2 and 4, a flow diagram associated with such a procedure 200 is shown. As indicated at 202, a guidewire (or other elongated device) is positioned within a patient's heart. At 204, pressures within the aorta and within the left ventricle are taken using pressure sensors located on the guidewire. At 206, the pressure data from the pressure sensors is displayed on a device for a practitioner to review. At 208, a valve is positioned, is in a TAVR/TAVI procedure using a catheter that is guided to the valve location by way of the guidewire (e.g., the guidewire is disposed within a lumen of the catheter to guide it to the desired location). At 210, pressures within the aorta and within the left ventricle are again taken using the pressures sensors of the guidewire. At 212, the observed pressures prior to placement of the new valve and pressures observed after the placement of the new valve are compared. At 214 a determination is made whether to make any adjustments to the valve. This determination may be made by comparing pressures taken pre-and post-placement of the valve, including comparison of actual pressures, comparison of pressure ratios (e.g., a ratio of aortic pressure to ventricular pressure—or the inverse), a comparison to data in the cloud—which may include the use of machine learning or artificial intelligence to compare like data and previously documented outcomes. If adjustments are desired, adjustments are performed as indicated at 216 and the procedure can return to the act of obtaining pressures as indicated at 210. This cycle may be repeated as necessary until the pressures (and/or pressure ratio(s)) are within a desired range (or is deemed acceptable in comparison with data in the cloud) indicating proper seating or the valve and acceptable levels of regurgitation, at which time the procedure may be completed as indicated at 218.

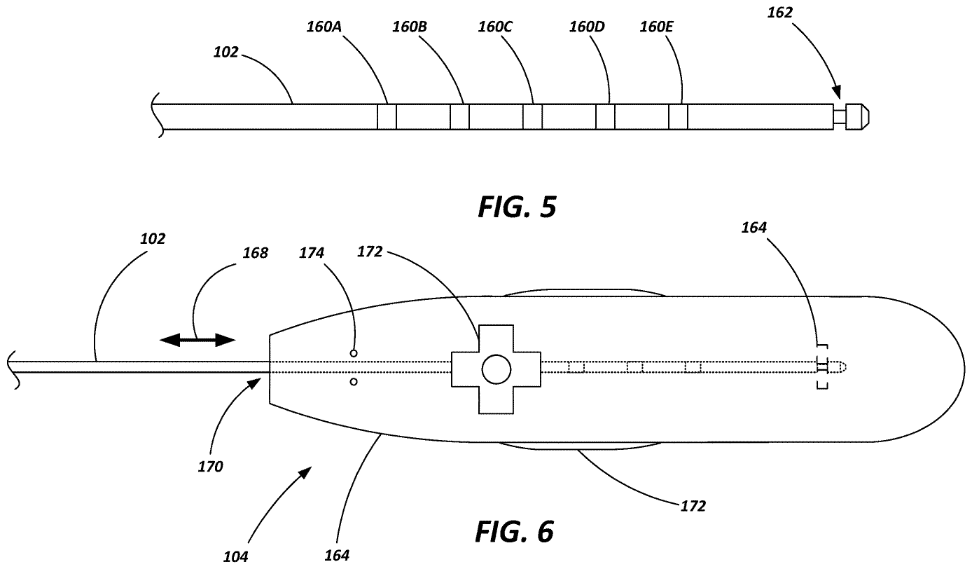

Referring now to FIGS. 5 and 6, a proximal end of the guidewire 102 and a control unit 104 for attachment to the proximal end of the guidewire 102, respectively, are shown. As shown in FIG. 5, a proximal end of the guidewire 102 may include a plurality of electrodes or electrical contacts 160A-160E. The contacts 160A-160E are configured to make electrical connection with corresponding contacts or electrodes (not shown) in the control unit 104. In the embodiment shown, each of the contacts 160A-160E may be electrically coupled with wires or conductors that extend a length of the guidewire 102 that are, in turn, electrically coupled with the sensors 106 in the distal section of the guidewire 102.

For example, two separate trifilar windings may be used to connect the sensors 106 to the contacts 160A-160E. In one embodiment, a first electrode 160A may be coupled with a power connection of a first sensor 106A using a first strand of a first trifilar winding, a second electrode 160B may be coupled with a data connection of a first sensor 106A using a second strand of a first trifilar winding, and a common connection may be made between the first sensor 106A and a third electrode 160C using a third strand of the first trifilar winding. Additionally, a fourth electrode 160D may be coupled with a power connection of a second sensor 106B using a first strand of a second trifilar winding, a fifth electrode 160E may be coupled with a data connection of a second sensor 106B using a second strand of a second trifilar winding, and a common connection may be made between the second sensor 106B and the third electrode 160C using a third strand of the second trifilar winding.

The guidewire 102 may additionally include a keyed or locking feature 162 in its proximal section. The locking feature 162 may include a shoulder or reduced diameter section configured for engagement with a locking structure or mechanism 164 located within a housing 166 of the control unit 104 (see FIG. 6). The locking feature 162 and locking mechanism 164 work together to retain the guidewire 102 in a desired position relative to the control unit 104 during use of the guidewire.

As indicated in FIG. 6, the guidewire 102 may be coupled with the control unit 104 by sliding the guidewire 102 in a direction parallel to its length (as indicated at 168) through an opening 170 formed in a surface of the housing 164 (FIG. 6 depicts the guidewire inserted into the housing as indicated by dashed lines). The control unit 104 (in addition to components and features previously mentioned) may include input features 172 (e.g., buttons, sliders, touchpads, directional pads, switches, etc.) to provide control of or communication with the sensors 106, transmission of data, and/or control of external components such as the external computing device 110 or monitor 112 (see FIG. 1). Additionally, the control unit 104 may include output features 174 (e.g., lights, screens, audio speakers, etc.) to provide feedback regarding operational status of the guidewire 102 or other components of the system 100.

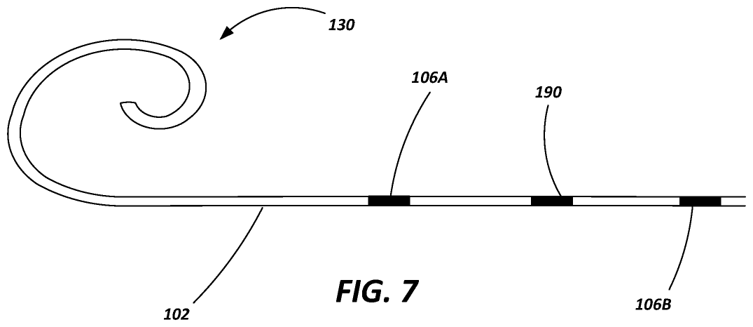

Referring now to FIG. 7, the distal end of a guidewire 102 is depicted in accordance with another embodiment of the present disclosure. The guidewire is generally constructed similarly to that which has been described previously herein. The guidewire 102 may include a pair of spaced apart sensors 106A and 106B used, for example, to simultaneously determine pressure in a left ventricle and an aorta, respectively, of a patient. The guidewire may additionally include an imaging sensor 190 longitudinally disposed between the two sensors 106A and 106B. In one embodiment, the imaging sensor may include an ultrasound transducer (or an array of ultrasound transducers) that enable imaging of for example, the aortic valve. In one example, the imaging sensor may be used to determine, for example, the size of a replacement valve that may need to be used. In other examples, the imaging sensor may be used to determine whether a new valve has been suitably positioned or whether regurgitation exists after initial placement. In one embodiment, such an imaging sensor 190 may include one or more transducers such as described in PCT Patent Application No. PCTUS2023/022337, entitled CMUT MEDICAL DEVICES, FABRICATION METHODS, SYSTEMS AND RELATED METHODS, and filed on May 16, 2023, the disclosure of which is incorporated by reference herein in its entirety. In another embodiment, the imagine sensor 190 may be constructed in a manner such as described in PCT Patent Application Publication No. WO2023/196559 entitled MEDICAL DEVICES, SENSORS FOR MEDICAL DEVICES AND RELATED METHODS, and filed on Apr. 7, 2023, the disclosure of which is incorporated by reference herein in its entirety. In one embodiment, the imaging sensor 190 may be positioned approximately an equal distance from each of pair of sensors 106A and 106B. In other embodiments, the imaging sensor 190 may be offset towards one sensor (106A) or the other sensor (106B).

In other embodiments, different numbers of and types of sensors may be used. For example, in some embodiments, ten or more pressures sensors may be used to determine a pressure gradient within a portion of a patient's vasculature. In some embodiments, one or more pressure sensors may be combined with one or more flow sensors to determine multiple physiological variables.

While the disclosed embodiments may be susceptible to various modifications and alternative forms, specific embodiments have been shown by way of example in the drawings and have been described in detail herein. It is noted that features, elements, or components of one embodiment may be combined with features, elements, or components of other embodiments without limitation. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical system comprising:
a guidewire having a pair of pressure sensors in a distal section of the guidewire;
a control unit selectively coupled with a proximal end of the guidewire;
an external computing device in wireless communication with the control unit;
a monitor in wireless communication with the external computing unit configured to display pressure data obtained by the pair of pressure sensors.

2. The medical system of claim 1, wherein the external computing device is in communication with a healthcare facility network.

3. The medical system of claim 2, wherein the external computing device is in communication with an external database.

4. The system of claim 3, wherein the pair of pressure sensors are spaced between approximately 9 centimeters (cm) and approximately 10 cm from each other in a center-to-center fashion.

5. The system of claim 4, wherein the guidewire includes a coiled section at a distal portion of the guidewire that extends from a longitudinal portion of the guidewire.

6. The system of claim 5, wherein a distal-most sensor of the pair of sensors is positioned a distance of between approximately 2 cm and approximately 2.5 cm from a line that is tangent to a proximal-most portion of the coiled section and is perpendicular to the longitudinal portion to the center of the distal-most sensor.

7. The system of claim 6, wherein the guidewire further includes an imaging device positioned longitudinally between the pair of pressure sensors.

8. The system of claim 7, wherein the imaging device includes an ultrasound transducer.

9. A medical device comprising:
a guidewire having a pair of pressure sensors in a distal section of the guidewire;
an imaging device associated with the guidewire, the imaging device positioned longitudinally between the pair of pressure sensors.

10. The medical device of claim 9, wherein the imaging sensor includes at least one ultrasound transducer.

11. The medical device of claim 5, wherein the pair of pressure sensors are spaced between approximately 9 centimeters (cm) and approximately 10 cm from each other in a center-to-center fashion.

12. The medical device of claim 11, wherein the guidewire includes a coiled section at a distal portion of the guidewire that extends from a longitudinal portion of the guidewire.

13. The medical device of claim 12, wherein a distal-most sensor of the pair of sensors is positioned a distance of between approximately 2 cm and approximately 2.5 cm from a line that is tangent to a proximal-most portion of the coiled section and is perpendicular to the longitudinal portion to the center of the distal-most sensor.

14. A method comprising:
positioning a guidewire in a patient such that a first sensor of the guidewire is positioned in a left ventricle and a second sensor is positioned in an aorta;
measuring a first aortic pressure with a first sensor and a first ventricular pressure with the second sensor;
positioning a replacement valve at a location between the first sensor and the second sensor;
measuring a second aortic pressure with the first sensor and a second ventricular pressure with the second sensor;
determining whether to adjust the replacement valve based on a comparison of data obtained from the first aortic pressure, the second aortic pressure, the first ventricular pressure, and the second ventricular pressure.

15. The method according to claim 14, further comprising adjusting the replacement valve.

16. The method according to claim 15, further comprising:
measuring a third aortic pressure with the first sensor and a third ventricular pressure with the second sensor; and
determining whether to further adjust the replacement valve based on a comparison of data obtained from the first aortic pressure, the second aortic pressure, the third aortic pressure, the first ventricular pressure, the second ventricular pressure, and the third aortic pressure.

17. The method according to claim 14, wherein measuring the second aortic pressure and the second ventricular pressure is performed without removing the second sensor from the left ventricle subsequent the measuring of the first aortic pressure and the second aortic pressure.

18. The method according to claim 14, wherein determining whether to adjust the replacement valve based on a comparison of pressure data obtained from the first aortic pressure, the second aortic pressure, the first ventricular pressure, and the second ventricular pressure includes comparing the pressure data to information in a database of previously-obtained data.

19. The method according to claim 14, further comprising determining a size of the replacement valve using an imaging device of the guidewire located between the first sensor and the second sensor.
